(19) United States
(12) Patent Application Publication
(10) Pub. No.: US 2025/0025141 A1
(43) Pub. Date: Jan. 23, 2025

(54) BIOPSY DEVICE WITH INTEGRATED DITHER FEATURE

(71) Applicant: Devicor Medical Products, Inc., Cincinnati, OH (US)

(72) Inventor: Mark A. GRAHAM, Mason, OH (US)

(21) Appl. No.: 18/908,936

(22) Filed: Oct. 8, 2024

Related U.S. Application Data
(63) Continuation of application No. PCT/US23/18485, filed on Apr. 13, 2023.
(60) Provisional application No. 63/335,839, filed on Apr. 28, 2022.

(51) Int. Cl. A61B 10/02

(52) U.S. Cl. CPC A61B 10/0275; A61B 2010/0208

(57) ABSTRACT

A biopsy device includes a body, a needle, a cutter, and a cutter drive assembly. Body includes an outer housing. The needle extends distally from the body. The cutter is configured to rotate and translate relative to the needle. The cutter drive assembly includes a cutter overmold secured to a portion of the cutter. The cutter overmold includes a threaded portion and one or more channels. The threaded portion is configured to drive translation of the cutter. The one or more channels are configured to drive rotation of the cutter. The outer housing defines a resilient member extending inwardly from an inner surface of the outer housing. The resilient member is configured to engage the cutter overmold to apply either a proximally oriented force or a distally oriented force to the cutter overmold.

### PRIORITY

This application claims priority to U.S. Provisional Application Ser. No. 63/335,839, entitled “Biopsy Device with Integrated Dither Feature,” filed on Apr. 28, 2022, the disclosure of which is incorporated by reference herein.

### BACKGROUND

Biopsy samples have been obtained in a variety of ways in various medical procedures using a variety of devices. Biopsy devices may be used under stereotactic guidance, ultrasound guidance, MRI guidance, PEM guidance, BSGI guidance, or otherwise. For instance, some biopsy devices may be fully operable by a user using a single hand, and with a single insertion, to capture one or more biopsy samples from a patient. In addition, some biopsy devices may be tethered to a vacuum module and/or control module, such as for communication of fluids (e.g., pressurized air, saline, atmospheric air, vacuum, etc.), for communication of power, and/or for communication of commands and the like. Other biopsy devices may be fully or at least partially operable without being tethered or otherwise connected with another device.

Examples of some versions of biopsy devices and biopsy system components are disclosed in U.S. Pat. No. 5,928,164, entitled “Apparatus for Automated Biopsy and Collection of Soft Tissue,” issued Jul. 27, 1999; U.S. Pat. No. 6,086,544, entitled “Control Apparatus for an Automated Surgical Biopsy Device,” issued Jul. 11, 2000; U.S. Pat. No. 6,752,768, entitled “Surgical Biopsy System with Remote Control for Selecting an Operational Mode,” issued Jun. 22, 2004; U.S. Pat. No. 7,442,171, entitled “Remote Thumbwheel for a Surgical Biopsy Device,” issued Oct. 8, 2008; U.S. Pat. No. 7,837,632, entitled “Biopsy Device Tissue Port Adjustment,” issued Nov. 23, 2010; U.S. Pat. No. 7,854,706, entitled “Clutch and Valving System for Tetherless Biopsy Device,” issued Dec. 1, 2010; U.S. Pat. No. 7,938,786, entitled “Vacuum Timing Algorithm for Biopsy Device,” issued May 10, 2011; U.S. Pat. No. 8,118,755, entitled “Biopsy Sample Storage,” issued Feb. 21, 2012; U.S. Pat. No. 8,206,316, entitled “Tetherless Biopsy Device with Reusable Portion,” issued Jun. 26, 2012; U.S. Pat. No. 8,241,226, entitled “Biopsy Device with Rotatable Tissue Sample Holder,” issued Aug. 14, 2012; and U.S. Pat. No. 9,345,457 entitled “Presentation of Biopsy Sample by Biopsy Device,” issued May 24, 2016. The disclosure of each of the above-cited U.S. patents is incorporated by reference herein.

Additional examples of versions of biopsy devices and biopsy system components are disclosed in U.S. Pub. No. 2010/0152610, entitled “Hand Actuated Tetherless Biopsy Device with Pistol Grip,” published Jun. 17, 2010; U.S. Pub. No. 2010/0160819, entitled “Biopsy Device with Central Thumbwheel,” published Jun. 24, 2010; U.S. Pub. No. 2012/0109007, entitled “Handheld Biopsy Device with Needle Firing,” published May 3, 2012; U.S. Pub. No. 2012/0283563, entitled “Biopsy Device with Manifold Alignment Feature and Tissue Sensor,” published Nov. 8, 2012; U.S. Pub. No. 2013/0324882, entitled “Control for Biopsy Device,” published Dec. 5, 2013; U.S. Pub. No. 2014/0039343, entitled “Biopsy System,” published Feb. 6, 2014; and U.S. Pub. No. 2018/0153529, entitled “Apparatus to Allow Biopsy Sample Visualization During Tissue Removal,” published Jun. 7, 2018. The disclosure of each of the above-cited U.S. patent Application Publications is incorporated by reference herein.

While several systems and methods have been made and used for obtaining a biopsy sample, it is believed that no one prior to the inventor has made or used the invention described in the appended claims.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 depicts a schematic view of an example of a biopsy system including a biopsy device and a vacuum control module;

FIG. 2 depicts a perspective view of a probe of the biopsy device of FIG. 1, a portion of a housing of the probe being removed to show a cutter drive assembly;

FIG. 3 depicts a perspective exploded view of the cutter drive assembly of FIG. 2;

FIG. 4 depicts a perspective view of a cutter overmold of the cutter drive assembly of FIG. 2;

FIG. 5 depicts a perspective view of a translation member of the cutter drive assembly of FIG. 2;

FIG. 6 depicts a front elevational view of a rotation member of the cutter drive assembly of FIG. 2;

FIG. 7 depicts a perspective cross-sectional view of the cutter drive assembly of FIG. 2, the cross-section taken along line 7-7 of FIG. 2;

FIG. 8 depicts a detailed perspective view of a portion of the housing of FIG. 2;

FIG. 9A depicts another perspective cross-sectional view of the cutter drive assembly of FIG. 2, the cutter drive assembly being used to translate the cutter distally;

FIG. 9B depicts yet another perspective cross-sectional view of the cutter drive assembly of FIG. 2, the cutter drive assembly being used to rotate the cutter while in a distal position;

FIG. 9C depicts still another perspective cross-sectional view of the cutter drive assembly of FIG. 2, the cutter drive assembly being used to translate the cutter proximally;

FIG. 9D depicts still another perspective cross-sectional view of the cutter drive assembly of FIG. 2, the cutter drive assembly being used to rotate the cutter while in a proximal position;

FIG. 10A depicts a top cross-sectional view of the cutter drive assembly of FIG. 2, the cutter drive assembly being used to translate the cutter distally;

FIG. 10B depicts another top cross-sectional view of the cutter drive assembly of FIG. 2, the cutter drive assembly being used to rotate the cutter while in the distal position; and

FIG. 10C depicts yet another top cross-sectional view of the cutter drive assembly of FIG. 2, the cutter drive assembly being used to rotate the cutter while in the proximal position.

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description serve to explain the principles of the technology; it being understood, however, that this technology is not limited to the precise arrangements shown.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

### I. OVERVIEW OF EXAMPLE OF BIOPSY SYSTEM

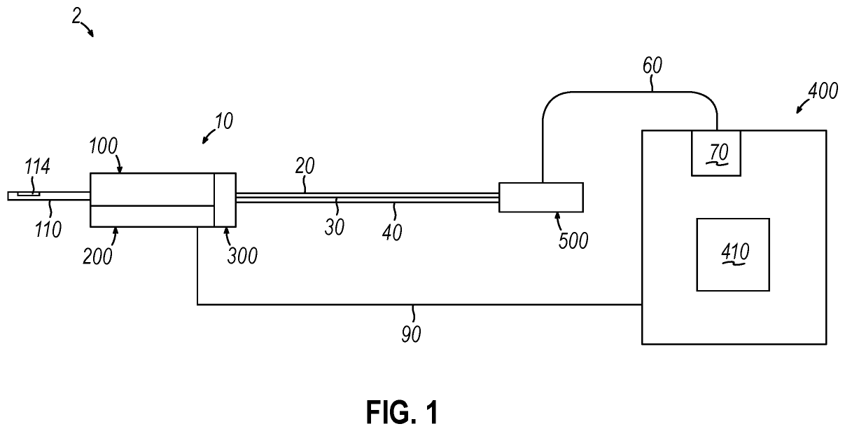

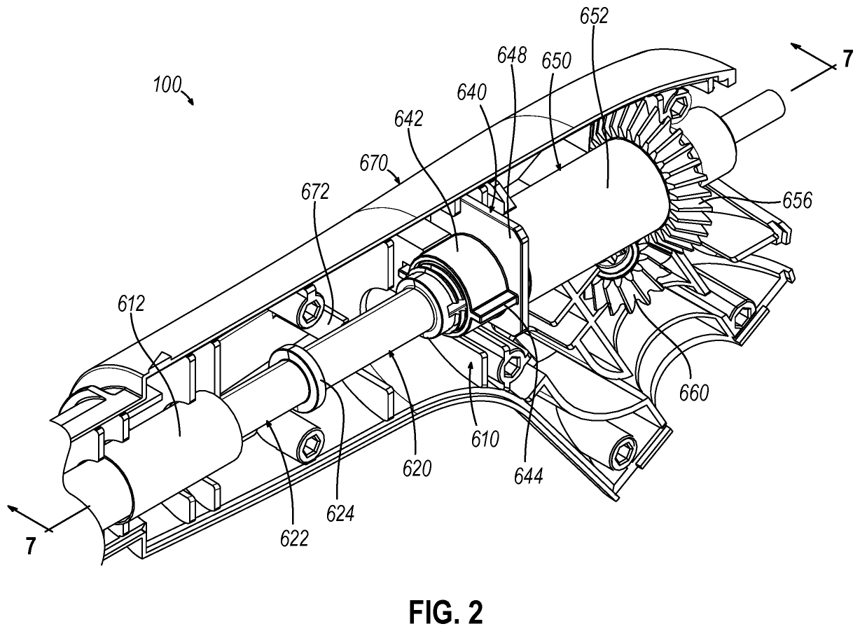

FIG. 1 shows aspects of a biopsy system (2), which may include a biopsy device (10) and a vacuum control module (400). Biopsy device (10) of this example includes a probe (100) and a holster (200). A needle (110) extends distally from probe (100), and may be insertable into a patient's tissue to obtain tissue samples by translating a hollow cutter (150) (see FIG. 2) relative to a lateral aperture (114). Such tissue samples may be transported through needle (110) via cutter (150) and deposited in a tissue sample holder (300) at the proximal end of probe (100), as will also be described in greater detail below.

It should also be understood that the use of the term “holster” herein should not be read as requiring any portion of probe (100) to be inserted into any portion of holster (200). For instance, in some versions, holster (200) may merely abut probe (100) with one or more prongs (not shown) being used to connect probe (100) to holster (200). In other versions, holster (200) may receive a portion of probe (100) to promote coupling between probe (100) and holster (200). In still other versions, probe (100) and holster (200) may be of unitary or integral construction, such that the two components cannot be separated. By way of example only, in versions where probe (100) and holster (200) are provided as separable components, probe (100) may be provided as a disposable component, while holster (200) may be provided as a reusable component. Still other suitable structural and functional relationships between probe (100) and holster (200) will be apparent to those of ordinary skill in the art in view of the teachings herein.

It should be understood that biopsy device (10) may be used in connection with a variety of imaging guidance modalities. Suitable imaging guidance modalities may include stereotactic guidance, ultrasound guidance, MRI guidance, and/or etc. In some versions, biopsy device (10) may include specific features and/or configurations specific to a given imaging guidance modality. For instance, with respect to stereotactic guidance, biopsy device (10) may be configured to mount to a table or fixture. For stereotactic guidance or other imaging guidance modalities, biopsy device (10) may be sized and configured such that biopsy device (10) may be operated by a single hand of an operator. In particular, an operator may grasp biopsy device (10), insert needle (110) into a patient's breast, and collect one or a plurality of tissue samples from within the patient's breast, all with just using a single hand. Alternatively, an operator may grasp biopsy device (10) with more than one hand and/or with any desired assistance. In some settings, the operator may capture a plurality of tissue samples with just a single insertion of needle (110) into the patient's breast. Such tissue samples may be pneumatically deposited in tissue sample holder (300), and later retrieved from tissue sample holder (300) for analysis. While examples described herein often refer to the acquisition of biopsy samples from a patient's breast, it should be understood that biopsy device (10) may be used in a variety of other procedures for a variety of other purposes and in a variety of other parts of a patient's anatomy (e.g., prostate, thyroid, etc.). Various exemplary components, features, configurations, and operabilities of biopsy device (10) will be described in greater detail below; while other suitable components, features, configurations, and operabilities will be apparent to those of ordinary skill in the art in view of the teachings herein.

Holster (200) of the present version is generally configured to drive and/or control one or more features of probe (100). For instance, holster (200) may include one or more motors (not shown), which may be in communication with probe (100) to drive movement of cutter (150) for acquisition of one or more tissue samples. In addition, or in the alternative, some versions of holster (200) may include one or more user input features to faciliate control of various functions of probe (100).

Holster (200) may include one or more drive gears (not shown), which may be in communication with probe (100) rotate and translate cutter (150), as will be described in greater detail below. In some versions, such gears may be driven by motors contained within holster (200) or by motors positioned outside of holster (200) but in communication with holster (200). Motors referred to herein may receive power from vacuum control module (400) via cable (90). In addition, data may be communicated between vacuum control module (400) and holster (200) via cable (90). In some versions, such data may be used by control module (400) to display certain graphical user interface screens on a touchscreen (410) integrated into control module (400). In some other versions, one or more motors are powered by one or more batteries located within holster (200) and/or probe (100). It should therefore be understood that, as with other components described herein, cable (90) is merely optional. As yet another merely illustrative variation, motors may be powered pneumatically, such that cable (90) may be substituted with a conduit communicating a pressurized fluid medium to holster (200). As still other merely illustrative variation, cable (90) may include one or more rotary drive cables that are driven by motors that are located external to holster (200). It should also be understood that two or three of the motors may be combined as a single motor. Other suitable ways in which various the motors may be driven will be apparent to those of ordinary skill in the art in view of the teachings herein.

As described above, probe (100) of the present example includes a needle (110) extending distally from probe (100) may be insertable into a patient's tissue to obtain tissue samples. As shown in FIG. 1, vacuum control module (400) is coupled with probe (100) via a valve assembly (500) and tubes (20, 30, 40, 60), which is operable to selectively provide vacuum, saline, atmospheric air, and venting to probe (100). The internal components of the valve assembly of the present example are configured and arranged as described in U.S. Pub. No. 2013/0218047, entitled “Biopsy Device Valve Assembly,” published Aug. 22, 2013, the disclosure of which is incorporated by reference herein.

As also described above, one or more drive gears in some versions of holster (200) may be used to communicate power to probe (100). Thus, to faciliate communication of power to probe (100), probe (100) may include one/or more gears or other drive elements. In some versions, such gears may be exposed relative to a portion of holster (200) such that the one or more gears of probe (100) may mesh with corresponding one or more gears of holster (200). Alternatively, in other versions, probe (100) may include exposed splined elements or other rotary communication features that may engage with corresponding features of holster (200). Of course, in still other versions, various suitable alternative power communication features may be used to communicate power between probe (100) and holster (200) as will be apparent to those of ordinary skill in the art in view of the teachings herein.

Needle (110) of the present version includes a lateral aperture (114) located proximate the distal end of needle (110). In some versions, the distal end of needle (110) may be of a blunt configuration. Such blunt configurations may be desirable in versions of biopsy device (10) where tissue penetration is penetration is primarilly performed using associated accessory components such as targeting sets used in MR guided procedures. Alternatively, the distal tip of needle (110) may include a sharp tissue piercing tip that may be configured to pierce and penetrate tissue. Other suitable configurations that may be used for the distal end of needle (110) will be apparent to those of ordinary skill in the art in view of the teachings herein.

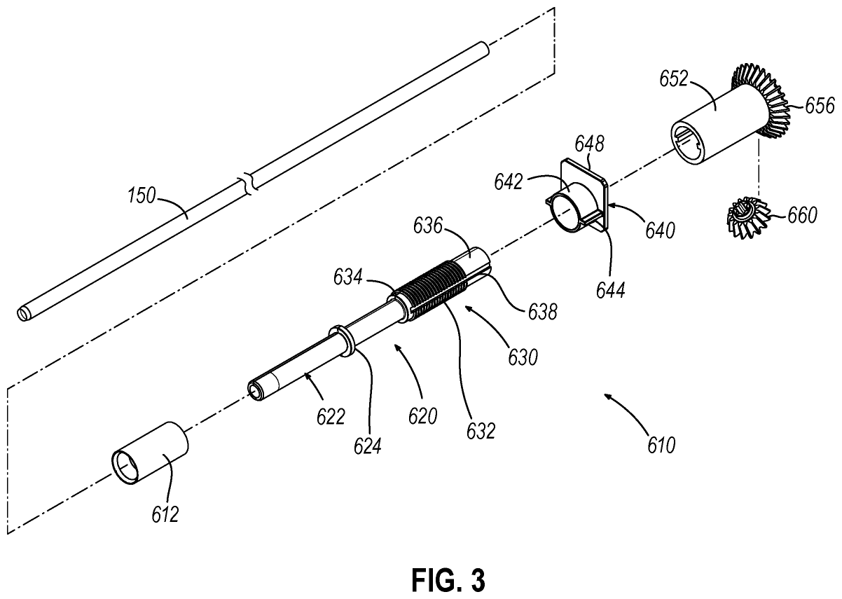

Lateral aperture (114) is sized to receive prolapsed tissue during operation of biopsy device (10). A hollow tubular cutter (150) (see FIGS. 2 and 3) having a sharp distal edge may be disposed within needle (110). Cutter (150) is operable to rotate and translate relative to needle (110) and past lateral aperture (114) to sever a tissue sample from tissue protruding through lateral aperture (114). For instance, cutter (150) may be moved from an extended position to a retracted position, thereby “opening” lateral aperture (114) to allow tissue to protrude therethrough; then from the retracted position back to the extended position to sever the protruding tissue.

Although not shown, it should be understood that in some versions, needle (110) may include a multi-lumen configuration. In such versions, an upper lumen (alternatively referred to as an axial lumen) may be used to receive cutter (150), while a lower lumen may be used to communicate atmosphere or other fluids to the distal end of needle (110). Such multi-lumen configurations may be facilitated in some versions with a longitudinal wall, walls, tubes, and/or other strcutural features. As will be appreciated, the presence of an additional lumen may be desirable to promote transport of one or more tissue samples though cutter (150) by promoting an optimum fluid pressure balance on each side of the tissue sample being transported. Suitable multi-lumen configurations for needle (110) are disclosed in U.S. Pat. No. 7,918,803, entitled “Methods and Devices for Automated Biopsy and Collection of Soft Tissue,” issued Apr. 5, 2011, the disclosure of which is incorporated by reference herein.

### II. EXAMPLE OF CUTTER DRIVE ASSEMBLY WITH INTEGRAL RETURN FEATURE

As noted above, cutter (150) is operable to simultaneously translate and rotate relative to needle (110) to sever a tissue sample from tissue protruding through lateral aperture (114). As best seen in FIGS. 2 through 7, probe (100) includes an outer housing (670), which contains and holds at least a portion of a cutter drive assembly (610). Cutter drive assembly (610) is configured to drive translation and rotation of cutter (150) relative to needle (110). As will be described in greater detail below, cutter drive assembly (610) is generally configured to receive rotary input from holster (200) or other suitable components to convert such a rotary input into translation and rotation of cutter (150). Although a single rotary input is used in the present version to drive cutter drive assembly (610), it should be understood that in other versions, cutter drive assembly (610) may be configured to receive multiple inputs of either rotary or linear modes of input.

As best seen in FIG. 3, cutter drive assembly (610) includes a manifold (612), a cutter overmold (620) (also referred to as a sleeve), a translation member (640), a rotation member (650) and a cutter drive gear (660). Although not shown, it should be understood that manifold (612) may be in fluid communication with a portion of needle (110) to promote fluid flow from one or more tubes (20, 30, 40, 60) into at least a portion of needle (110). To faciliate such fluid flow, while also permitting movement of cutter (150), manifold (612) may receive at least a portion of cutter (150) and/or cutter overmold (620), while permitting translation and rotation of cutter (150) and/or cutter overmold (620) relative to manifold (612).

Cutter overmold (620) is generally configured to convert rotary input from cutter drive gear (660) into rotation and translation of cutter (150) in cooperation with translation member (640) and rotation member (650). Thus, cutter overmold (620) is generally overmolded or otherwise secured to an outer surface of cutter (150) such that rotation and/or translation of cutter overmold (620) will result in corresponding rotation and/or translation of cutter (150). Although overmolding is used in the present version to secure cutter overmold (620) to cutter (150) it should be understood that in other versions, any suitable fastening mechanism may be used such as adhesives, mechanical fasteners, and/or etc.

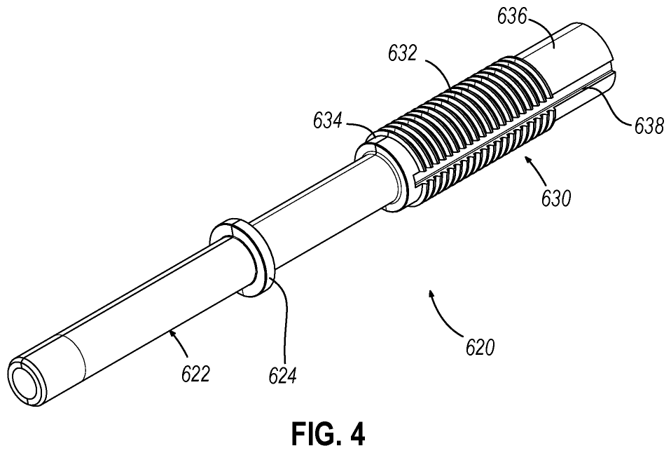

As best seen in FIG. 4, cutter overmold (620) includes a generally smooth and cylindraceous distal portion (622) and a drive portion (630). Distal portion (622) is generally configured for receipt within manifold (612) such that distal portion (622) may seal against the inner diameter of manifold (612). Thus, at least a portion of distal portion (622) may be configured to seal manifold (612) to promote the flow of fluid within manifold (612) and into needle (110).

Distal portion (622) further defines a flange (624) disposed at about the center of distal portion (622). As will be described in greater detail below, flange (624) may be used in some versions to set a proximal position of cutter overmold (620) and/or cutter (150) relative to other components of cutter drive assembly (610). Optionally, in some versions, flange (624) may additionally used in connection with manifold (612) to drive valve components within manifold (612) or otherwise manipulate sealing between cutter overmold (620), and manifold (612).

Drive portion (630) is generally configured to engage translation member (640) and rotation member (650) to drive translation and rotation of cutter (150) in response to rotary input from rotation member (650). Drive portion (630) includes threading (632) and one or more channels (638) (also referred to as keyed portion). Threading (632) is generally configured to engage a complementary feature of translation member (640) to drive translation of cutter overmold (620) and cutter (150) using rotation of cutter overmold (620). Threading (632) is disposed between a distal zero pitch portion (634) and a proximal zero pitch portion (636). As will be described in greater detail below, the combination of threading (632) and zero pitch portions (634, 636) is configured to provide translation of cutter (150) through a predetermined range of motion, while still permitting cutter (150) to rotate outside of the predetermined range of motion.

One or more channels (638) are generally configured to engage rotation member (650) to communicate rotary movement of rotation member (650) to cutter overmold (620) and cutter (150). Each channel (638) extends into the outer diameter of drive portion (630) to define a rectangular cross-sectional area for receipt of a corresponding feature of rotation member (650). In other words, each channel (638) is defined by a slot extending from one end of drive portion (630) to another. Although only one channel (638) is visible in FIG. 4, it should be understood that the present version may include another substantially similar channel (638) on the opposite side of drive portion (630). Alternatively, in some versions, other suitable numbers of channels (638) may be used such as three, four, or etc. In still other versions, one or more channels (638) may be omitted entirely and replaced with other strcutural features such as protrusions, or unique geometric shapes such as a hexagonally-shaped rod.

Each channel (638) of the present version overlaps with threading (632) such that each individual thread of threading (632) is interrupted by a portion of each channel (638). Each channel (638) further extends into at least a portion of each zero pitch portion (634, 636) to permit cutter overmold (620) to be rotated continuously regardless of the particular relative translation position of cutter overmold (620). The particular overlapping configuration of the present version may be desirable in some circumstances where the size of probe (100) is constrained. For instance, by using the overlapping configuration of the present version, the overall length of cutter drive assembly (610) may be reduced. In other versions, threading (632) and channels (638) (or other keyed features) may be separate from each other, thus providing a separate section of cutter overmold (620) to engage translation member (640) and another separate section to engage rotation member (650). Such an alternative version may be desirable to promote ease of manufacturability in circumstances where the size of probe (100) is not constrained.

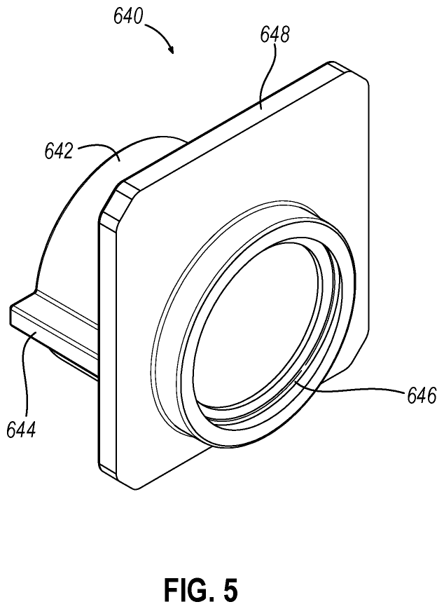

Translation member (640) is generally configured to receive a portion of cutter overmold (620) to engage threading (632) and convert rotation of cutter overmold (620) into translation of cutter overmold (620). As best seen in FIG. 5, translation member (640) includes a receiver (642) and a base (648), with a portion of receiver (642) extending from a proximal and distal side of base (648). Receiver (642) is generally configured to receive at least a portion of drive portion (630) of cutter overmold (620) coaxially. Thus, receiver (642) defines a generally cylindrical shape corresponding to the shape of drive portion (630).

The exterior of receiver (642) includes one or more wings (644) extending from an outer surface of receiver (642). Wings (644) are positioned on a distal side of receiver (642), distally of base (648). Each wing (644) is generally configured to engage a portion of probe (100) to anchor the rotation position of translation member (640). In other words, each wing (644) is generally configured to prevent or resist rotation of translation member (640) relative to cutter overmold (620). Although the present version includes two wings (644), it should be understood that in other versions any suitable number of wings (644) may be used. In still other versions, wings (644) may be omitted entirely and instead be replaced with other mechanical grounding features such as posts, legs, or the outer diameter of receiver (642) being of an irregular shape.

The inner diameter of receiver (642) defines one or more threads (646) extending around the inner diameter of receiver (642). Threads (646) are generally configured to engage threading (632) of drive portion (630). As will be described in greater detail below, translation member (640) may be rotationally stationary relative to cutter overmold (620). Thus, threads (646) may be used to drive translation of cutter overmold (620) via engagement with threading (632) in response to rotation of cutter overmold (620) relative to translation member (640). In the present version, threads (646) are isolated to the proximal end of receiver (642). However, it should be understood that in other versions, threads (646) may be disposed at any suitable position along the inner diameter of receiver (642) or alternatively extend through the entire length or receiver (642).

Base (648) extends outwardly from the outer diameter of receiver (642) and defines a square or rectangular-shaped profile. As will be described in greater detail below, base (648) may be configured to engage a portion of probe (100) to ground the axial position of translation member (640) within probe (100). Additionally, base (648) may also be used to prevent at least some rotation of translation member (640) within probe (100) as similarly described above with respect to wings (644).

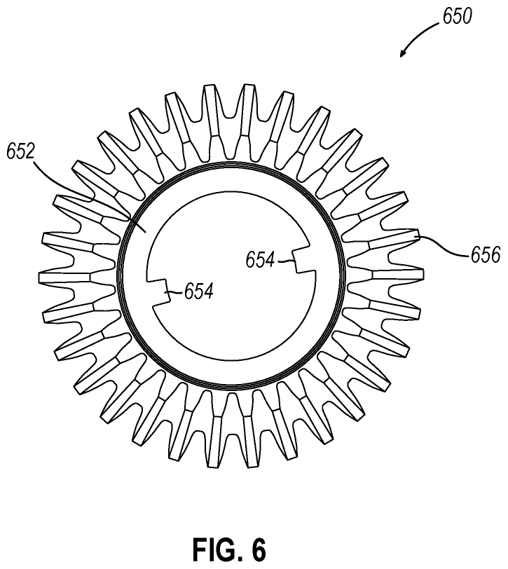

Rotation member (650) is generally configured to engage a portion of drive portion (630) to rotate cutter overmold (620). As best seen in FIG. 6, rotation member (650) includes a receiver (652) and a rotation gear (656). Receiver (652) is configured to receive at least a portion of drive portion (630) to drive rotation of cutter overmold (620). Thus, receiver (652) defines a generally hollow cylindrical shape corresponding to the shape of drive portion (630) of cutter overmold (620).

The inner diameter of receiver (652) includes one or more engagement protrusions (654) projecting into the hollow interior of receiver (652). Protrusions (654) are configured to engage channels (638) of drive portion (630). Thus, protrusions (654) may be used to mechanically ground rotation of rotation member (650) with rotation of cutter overmold (620). Two protrusions (654) are used in the present version that correspond to the square or rectangular shape of each corresponding channel (638). However, it should be understood that in other versions where different channel (638) configurations are used, different corresponding protrusion (654) configurations may likewise be used. In other words, protrusions (654) and channels (638) together define a keyed arrangement with a key-keyway configuration. Thus, it should be understood that in other versions, various alternative keyed configurations may be used that may be configured to transfer rotation from rotation member (650) to cutter overmold (620).

Rotation gear (656) extends from the outer diameter of receiver (652) at a proximal end of receiver (652). Rotation gear (656) is configured to mesh with drive gear (660) to receive rotation from drive gear (656) and rotate receiver (652). In the present version, rotation gear (656) is configured as a bevel gear so that drive gear (660) may be positioned at an angle relative to rotation gear (656). In other versions, rotation gear (656) may be configured as a spur gear or any other suitable gear to promote different orientations of drive gear (660). Additionally, although only drive gear (660) and rotation gear (656) are shown in the present version, it should be understood that in other version, additional gears may be used.

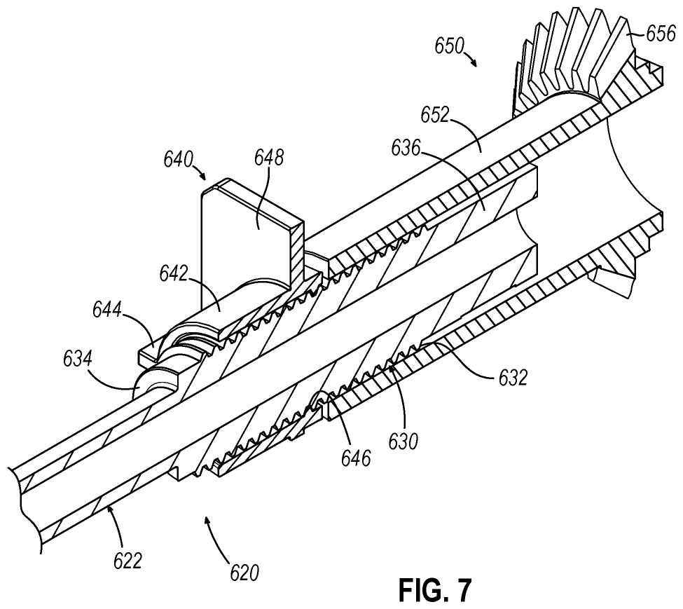

As best seen in FIG. 7, cutter overmold (620) may be received within the interior of both translation member (640) and rotation member (650). In particular, translation member (640) and rotation member (650) may be both disposed along a common axis corresponding to the longitudinal axis of cutter (150) and cutter overmold (620). Translation member (640) and rotation member (650) are generally proximate or adjacent relative to each other in the present version but may be spaced apart in other versions. The particular position of translation member (640) relative to rotation member (650) is such that translation member (640) may engage threading (632) of cutter overmold (620), while rotation member (650) may engage channels (638). Thus, translation member (640) is disposed distally of rotation member (650) in the present version due to the particular configuration of cutter overmold (150). However, it should be understood that the position of translation member (640) and rotation member (650) may be reversed in some versions.

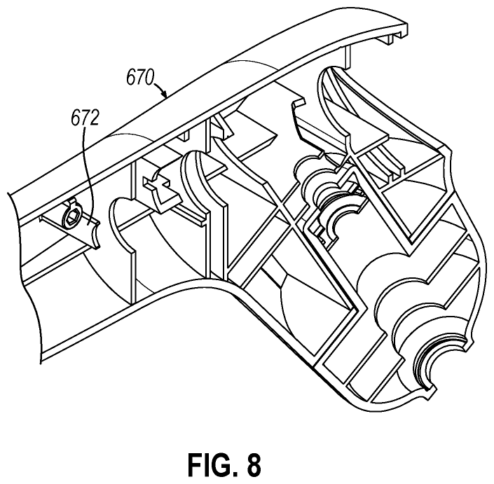

FIG. 8 shows outer housing (670) of probe (100) with the components of cutter drive assembly (610) removed to show the inner geometry of outer housing (670) in greater detail. As can be seen, outer housing (670) includes various shaped portions to hold components of cutter drive assembly (610) in a predetermined position. In addition, outer housing (670) includes one or more resilient members (672) configured to act as an integral return feature via active engagement with at least a portion of cutter drive assembly (610), as will be described in greater detail below.

Resilient member (672) projects inwardly from the outer surface defined by outer housing (670). The shape of the projection of resilient member (672) is generally rectangular with a concave curvature on the outer end of resilient member (672). As will be described in greater detail below, the concave curvature at the outer end of resilient member (672) is generally configured to engage certain components of cutter overmold (620). Although a generally rectangular shape is used in the present version, it should be understood that a variety of other shapes may be used such as square-shaped, triangle-shaped, oval-shaped, and/or etc.

Resilient member (672) is configured as a thin strip of integral construction with outer housing (670). The particular thickness of resilient member (672) may be a function of both the particular material of outer housing (670) and the thickness of resilient member (672). For instance, resilient member (672) is generally configured to be relatively still or rigid to resist movement of a portion of cutter drive assembly (610); yet resilient member (672) is also generally configured to provide some flexion in response to a portion of cutter drive assembly (610) to store potential energy within resilient member (672) from cutter drive assembly (610). In other words, resilient member (672) may be configured to have generally resilient or spring-like properties. Thus, resilient member (672) may have a variety of thicknesses depending on the mechanical properties of the material used for outer housing (670).

Although FIG. 8 shows only a single resilient member (672), it should be understood that the present version includes 2 resilient members (672). For instance, in the present version, outer housing (670) is of a claim shell configuration such that the version shown is one half of two generally mirrored halves. Thus, outer housing (670) may include another half that likewise includes a resilient member (672) substantially similar to resilient member (672) as shown.

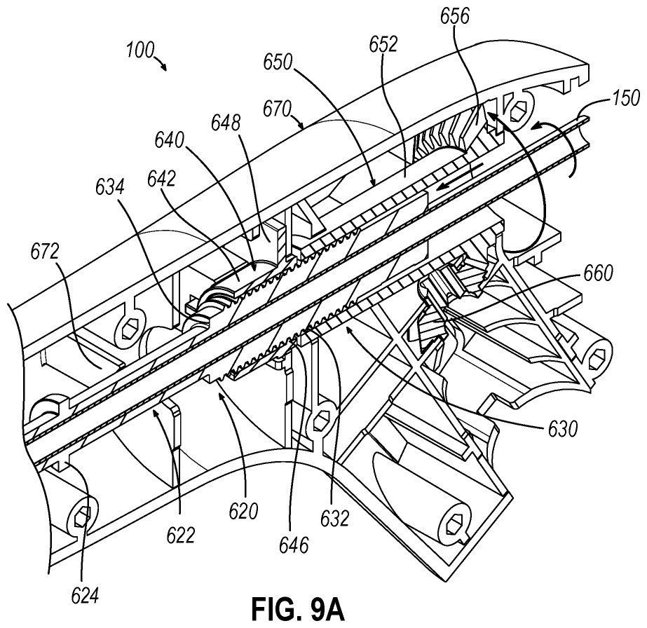

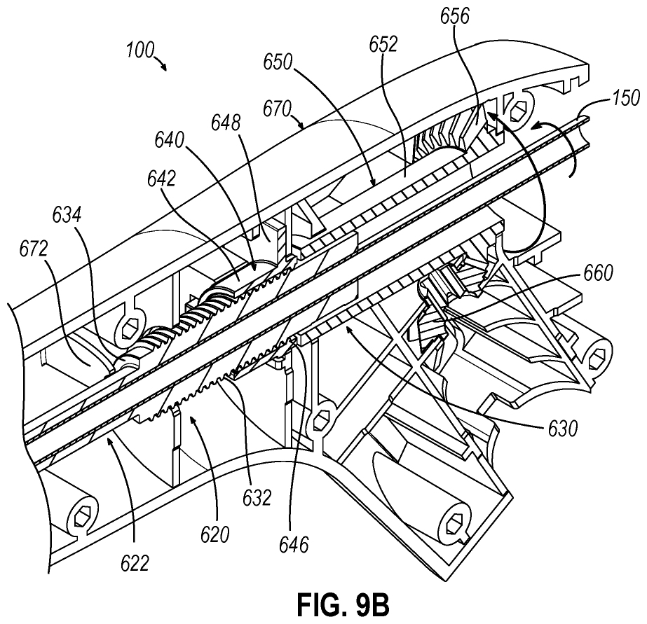

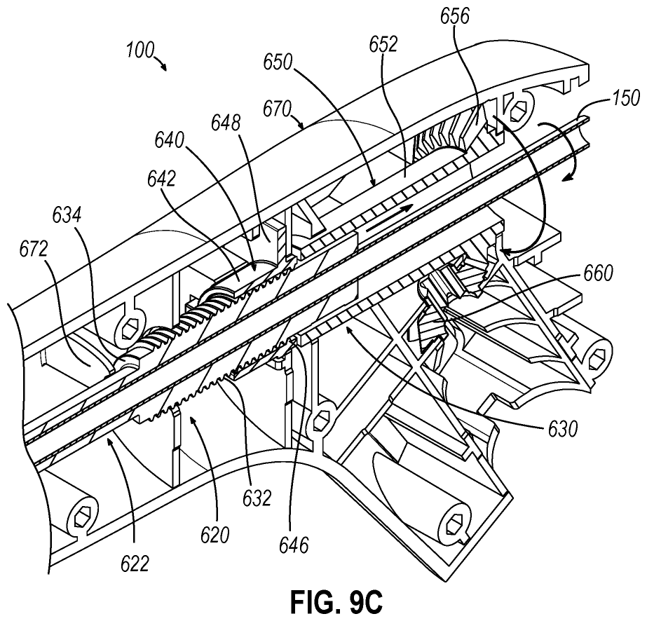

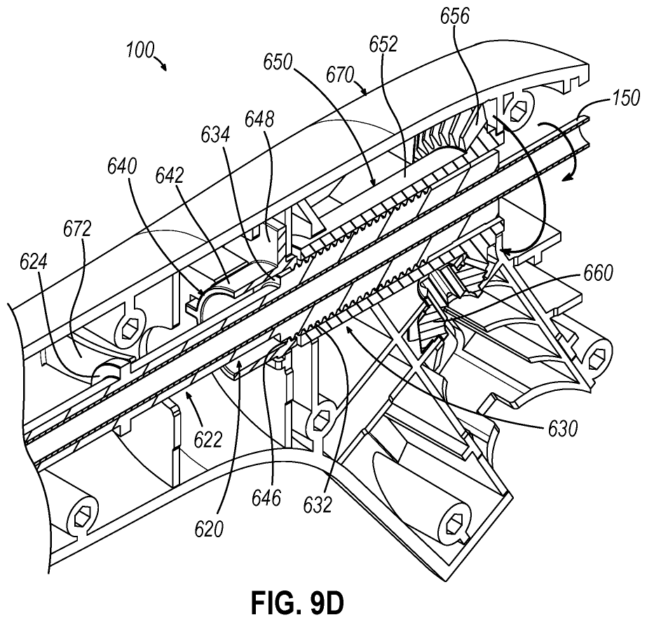

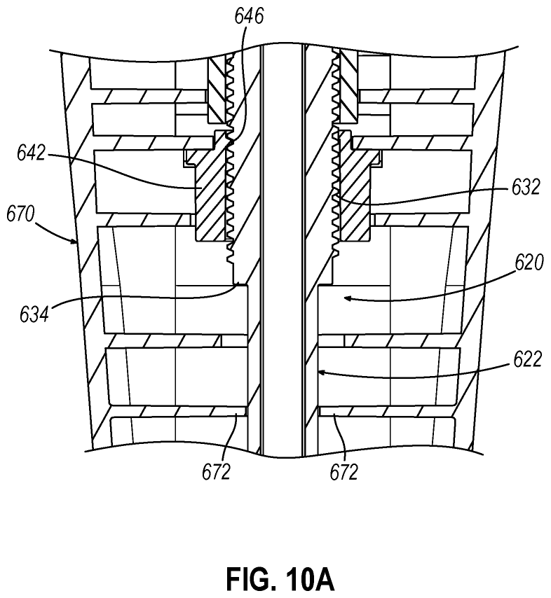

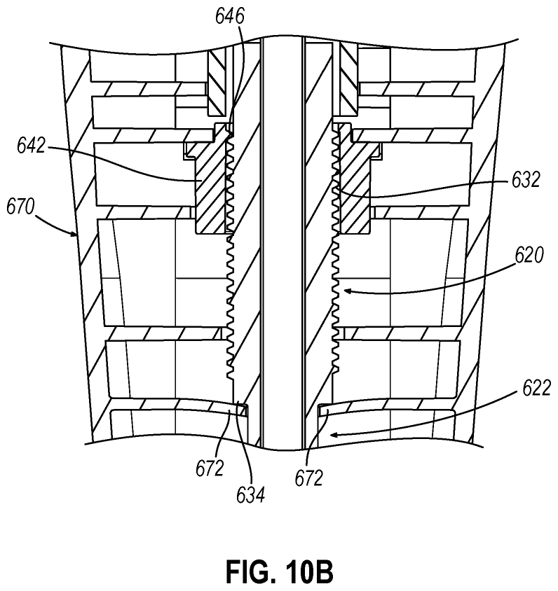

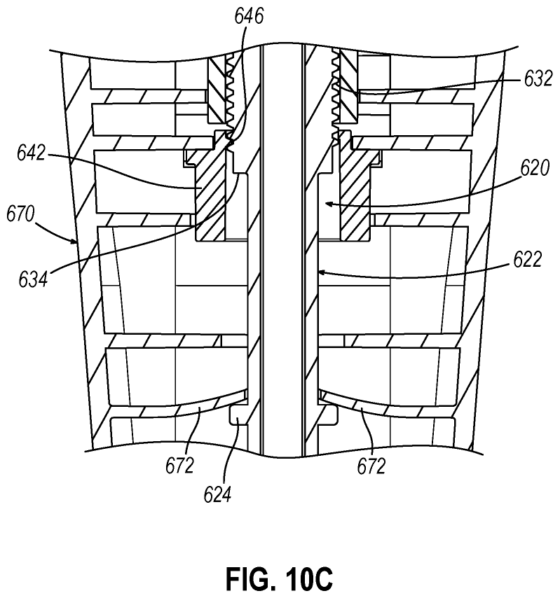

FIGS. 9A through 10C show an exemplary use of cutter drive assembly (610) in communication with resilient member (672) of outer housing (670). As can be seen in FIGS. 9A and 10A, cutter drive assembly (610) may initially be used to translate cutter (150) while cutter (150) is also rotated. In this configuration, drive gear (660) is rotated in a first direction to drive rotation of rotation member (650) in a first direction (e.g., counterclockwise as shown in FIG. 9A) via rotation gear (656).

Rotation of rotation member (650) may result in corresponding rotation of cutter overmold (610) and cutter (150) via engagement between engagement protrusions (654) and channels (638). Rotation of cutter overmold (610) may then also result in distal translation of cutter overmold (610) and cutter (150) via engagement between threads (646) of translation member (640) and threading (632) of cutter overmold (620).

As cutter (150) is being rotated and translated via cutter overmold (610) as shown in FIGS. 9A and 10A, resilient member (672) may remain adjacent to cutter overmold (620) but disengaged from cutter overmold (610) due to the configuration of distal portion (622). In other words, cutter overmold (610) may rotated and translate through a predetermined range of motion without engaging resilient member (672).

Simultaneous translation and rotation of cutter (150) via cutter overmold (620) may continue until threading (632) of cutter overmold (620) disengages from threads (646) of translation member (640). In other words, translation of cutter overmold (620) may continue until translation member (640) reaches proximal zero pitch portion (636) of cutter overmold (620). At this point, cutter overmold (620) and cutter (150) may continue to rotate via rotation member (650) without further translation due to the lack of threading on proximal zero pitch portion (636). In some circumstances, this state may be referred to as “free-wheeling.”

As threads (646) of translation member (640) approach proximal zero pitch portion (636), resilient member (672) may engage distal zero pitch portion (634) of cutter overmold (620) as shown in FIGS. 9B and 10B. Further translation of cutter overmold (620) may cause at least some bending or flexion of resilient member (672) as resilient member (672) absorbs translational energy from cutter overmold (620). As a result, resilient member (672) may exert a force in the proximal direction on cutter overmold (620).

With resilient member (672) engaged with cutter overmold (620) as shown in FIGS. 9B and 10B, at least some engagement between threads (646) of translation member (640) and threading (632) of cutter overmold (620) may be maintained by the proximal force applied by resilient member (672). During this state of partial engagement, cutter overmold (620) may still be freely rotatable. Additionally, some minor oscillating or back-and-forth translation of cutter overmold (620) and cutter (150) may occur as the ends of threads (646) and threading (632) interact with each other.

After distal translation of cutter (150) is complete, it may be desirable to reverse the translation of cutter (150) from a distal direction to a proximal direction. For instance, distal translation of cutter (150) may be used to close lateral aperture (114) to sever a tissue sample, while proximal translation of cutter (150) may be used to open lateral aperture (114) to receive tissue. As shown in FIG. 9C, the translation direction of cutter (150) may be reversed by reversing the rotation direction of drive gear (660) from the first direction described above to a second, opposite, direction. As a result, rotation member (650) may likewise reverse in an opposite second direction (e.g., clockwise as shown in FIG. 9C).

Upon reversal of rotation of rotation member (650), the force generated by resilient member (672) may be beneficial to promote full engagement between threading (632) of cutter overmold (620) and threads (646) of translation member (640). In particular, as noted above, resilient member (672) may provide a proximal force to cutter overmold (620) when engaged with distal zero pitch portion (634) of cutter overmold (620). With this proximal force applied, threading (632) and threads (646) may readily reengage upon reversal of the rotation of cutter overmold (620) via rotation member (650).

Upon reengagement of threading (632) and threads (646), cutter overmold (620) and cutter (150) may rotate and translate simultaneously through a predetermined range of proximal translation. As best seen in FIGS. 9D and 10C, proximal translation of cutter overmold (620) and cutter (150) may continue until threads (646) of translation member (640) reach the distal end of threading (632) of cutter overmold (620). In other words, proximal translation of cutter overmold (620) may continue until threads (646) of translation member (640) reach distal zero pitch portion (634) of cutter overmold (620). At this point, threading (632) and threads (646) may at least partially disengage from each other such that continued rotation of rotation member (650) results in rotation of cutter overmold (620).

As threads (646) of translation member (640) approach distal zero pitch portion (634), resilient member (672) may engage flange (624) of cutter overmold (620) as shown in FIGS. 9D and 10C. It should be thus understood that there may be a predetermined relationship between the position of flange (624), distal zero pitch portion (634), and the length of threading (632). In the present version, flange (624) is separated from distal zero pitch portion (634) by about a length corresponding to the length of threading (632). Further translation of cutter overmold (620) may cause at least some bending or flexion of resilient member (672) as resilient member (672) absorbs translational energy from cutter overmold (620). As a result, resilient member (672) may exert a force in the distal direction on cutter overmold (620).

With resilient member (672) engaged with flange (624) of cutter overmold (620) as shown in FIGS. 9D and 10C, at least some engagement between threads (646) of translation member (640) and threading (632) of cutter overmold (620) may be maintained by distal force applied by resilient member (672). During this state of partial engagement, cutter overmold (620) may still be freely rotatable. Additionally, some minor oscillating or back-and-forth translation of cutter overmold (620) and cutter (150) may occur as the ends of threads (646) and threading (632) interact with each other.

After proximal translation of cutter (150) is complete, it may be desirable to reverse the translation of cutter (150) from the proximal direction back to the distal direction. The translation direction of cutter (150) may be reversed as similarly described above by reversing the rotation direction of drive gear (660) from the second direction described above to the first direction described above. As a result, rotation member (650) may likewise reverse in the first direction.

Upon reversal of rotation of rotation member (650), the force generated by resilient member (672) may be beneficial to promote full engagement between threading (632) of cutter overmold (620) and threads (646) of translation member (640). In particular, as noted above, resilient member (672) may provide a distal force to cutter overmold (620) when engaged with flange (624) of cutter overmold (620). With this distal force applied, threading (632) and threads (646) may readily reengage upon reversal of the rotation of cutter overmold (620) via rotation member (650).

### III. EXAMPLE COMBINATIONS

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A biopsy device, the biopsy device comprising: a body including an outer housing; a needle extending distally from the body; a cutter configured to rotate and translate relative to the needle; and a cutter drive assembly, the cutter drive assembly including a cutter overmold secured to a portion of the cutter, the cutter overmold including a threaded portion and one or more channels, the threaded portion being configured to drive translation of the cutter, the one or more channels being configured to drive rotation of the cutter, the outer housing defining a resilient member extending inwardly from an inner surface of the outer housing, the resilient member being configured to engage the cutter overmold to apply either a proximally oriented force or a distally oriented force to the cutter overmold.

### Example 2

The biopsy device of Example 1, the resilient member being configured to apply both the proximally oriented force and the distally force to the cutter overmold.

### Example 3

The biopsy device of Example 2, the resilient member being configured to apply the proximally oriented force or the distally force to the cutter overmold depending on the orientation of the cutter overmold relative to the resilient member.

### Example 4

The biopsy device of any of Examples 1 through 3, the cutter overmold defining a distal zero pitch portion and a proximal zero pitch portion, the threaded portion being disposed between the distal zero pitch portion and the proximal zero pitch portion.

### Example 5

The biopsy device of Example 4, the resilient member being configured to engage the distal zero pitch portion.

### Example 6

The biopsy device of any of Examples 1 through 5, the resilient member being a first resilient member, the outer housing further defining a second resilient member, the first resilient member and the second resilient member extending inwardly from the inner surface of the outer housing in opposite directions.

### Example 7

The biopsy device of any of Examples 1 through 6, the cutter drive assembly further including a translation member and a rotation member, the translation member being configured to engage the threaded portion of the cutter overmold, the rotation member being configured to engage the one or more channels of the cutter overmold.

### Example 8

The biopsy device of Example 7, the rotation member being further configured to receive a single rotary input to drive simultaneous rotation and translation of the cutter.

### Example 9

The biopsy device of Examples 7 or 8, the translation member including one or more threads being configured to engage the threaded portion of the cutter overmold.

### Example 10

The biopsy device of Example 9, the resilient member being configured to maintain at least partial engagement between the one or more threads of the translation member and threaded portion of the cutter overmold during reversal of a translation direction of the cutter.

### Example 11

The biopsy device of Example 9, the resilient member being configured to maintain at least partial engagement between the one or more threads of the translation member and threaded portion of the cutter overmold during reversal of translation of the cutter from a distal direction to a proximal direction and during reversion of translation of the cutter from the proximal direction to the distal direction.

### Example 12

The biopsy device of any of Examples 1 through 11, the resilient member being configured to disengage from the cutter overmold for a predetermined range of cutter translation.

### Example 13

The biopsy device of any of Examples 1 through 12, the cutter overmold being configured to freely rotate relative to the resilient member.

### Example 14

The biopsy device of any of Examples 1 through 13, the cutter overmold further including a flange, the resilient member being configured to engage the flange.

### Example 15

The biopsy device of Example 14, the flange being disposed distally of the threaded portion.

### Example 16

A probe for use with a biopsy device, the probe comprising: a housing; a needle extending distally from the housing; a cutter configured to rotate and translate relative to the needle; and a cutter drive assembly, the cutter drive assembly including a sleeve coaxial with the cutter and configured to drive rotation and translation of the cutter, the sleeve including a threaded portion and a keyed portion, the threaded portion being configured to drive translation of the cutter, the keyed portion being configured to drive rotation of the cutter, a portion of the housing extending into a hollow interior of the outer housing to engage at least a portion of the sleeve when the sleeve is in a distal position and a proximal position.

### Example 17

The probe of Example 16, the portion of the housing extending into the hollow interior of the outer housing including an elongate strip, the elongate strip being configured to bend in response to movement of the sleeve within the housing.

### Example 18

The probe of Example 17, the sleeve including a flange and a distal zero pitch portion, the elongate strip being configured to alternatingly engage the flange and the distal zero pitch portion to maintain engagement between the threaded portion and a portion of the cutter drive assembly.

### Example 19

The probe of Example 18, the flange and the distal zero pitch portion being separated by a first length, the threaded portion defining a second length, the first length corresponding to the second length.

### Example 20

A probe for use with a biopsy device, the probe comprising: a housing; a needle extending distally from the housing; a cutter configured to rotate and translate relative to the needle; a cutter drive assembly, the cutter drive assembly including a sleeve coaxial with a longitudinal axis defined by the cutter and configured to drive rotation and translation of the cutter, the sleeve including a threaded portion and a keyed portion, the threaded portion being configured to drive translation of the cutter, the keyed portion being configured to drive rotation of the cutter; and a reversing mechanism mechanically grounded to a portion of the housing, the reversing mechanism being configured to bias the sleeve in a predetermined direction parallel to the longitudinal axis of the cutter.

### IV. CONCLUSION

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Embodiments of the present invention have application in conventional endoscopic and open surgical instrumentation as well as application in robotic-assisted surgery.

By way of example only, embodiments described herein may be processed before surgery. First, a new or used instrument may be obtained and if necessary cleaned. The instrument may then be sterilized. In one sterilization technique, the instrument is placed in a closed and sealed container, such as a plastic or TYVEK bag. The container and instrument may then be placed in a field of radiation that can penetrate the container, such as gamma radiation, x-rays, or high-energy electrons. The radiation may kill bacteria on the instrument and in the container. The sterilized instrument may then be stored in the sterile container. The sealed container may keep the instrument sterile until it is opened in a medical facility. A device may also be sterilized using any other technique known in the art, including but not limited to beta or gamma radiation, ethylene oxide, or steam.

Embodiments of the devices disclosed herein can be reconditioned for reuse after at least one use. Reconditioning may include any combination of the steps of disassembly of the device, followed by cleaning or replacement of particular pieces, and subsequent reassembly. In particular, embodiments of the devices disclosed herein may be disassembled, and any number of the particular pieces or parts of the devices may be selectively replaced or removed in any combination. Upon cleaning and/or replacement of particular parts, embodiments of the devices may be reassembled for subsequent use either at a reconditioning facility, or by a surgical team immediately prior to a surgical procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

1. A biopsy device, the biopsy device comprising:
(a) a body including an outer housing;
(b) a needle extending distally from the body;
(c) a cutter configured to rotate and translate relative to the needle; and
(d) a cutter drive assembly, the cutter drive assembly including a cutter overmold secured to a portion of the cutter, the cutter overmold including a threaded portion and one or more channels, the threaded portion being configured to drive translation of the cutter, the one or more channels being configured to drive rotation of the cutter,
the outer housing defining a resilient member extending inwardly from an inner surface of the outer housing, the resilient member being configured to engage the cutter overmold to apply either a proximally oriented force or a distally oriented force to the cutter overmold.

2. The biopsy device of claim 1, the resilient member being configured to apply both the proximally oriented force and the distally oriented force to the cutter overmold.

3. The biopsy device of claim 2, the resilient member being configured to apply the proximally oriented force or the distally oriented force to the cutter overmold depending on the orientation of the cutter overmold relative to the resilient member.

4. The biopsy device of claim 1, the cutter overmold defining a distal zero pitch portion and a proximal zero pitch portion, the threaded portion being disposed between the distal zero pitch portion and the proximal zero pitch portion.

5. The biopsy device of claim 4, the resilient member being configured to engage the distal zero pitch portion.

6. The biopsy device of claim 1, the resilient member being a first resilient member, the outer housing further defining a second resilient member, the first resilient member and the second resilient member extending inwardly from the inner surface of the outer housing in opposite directions.

7. The biopsy device of claim 1, the cutter drive assembly further including a translation member and a rotation member, the translation member being configured to engage the threaded portion of the cutter overmold, the rotation member being configured to engage the one or more channels of the cutter overmold.

8. The biopsy device of claim 7, the rotation member being further configured to receive a single rotary input to drive simultaneous rotation and translation of the cutter.

9. The biopsy device of claim 7, the translation member including one or more threads being configured to engage the threaded portion of the cutter overmold.

10. The biopsy device of claim 9, the resilient member being configured to maintain at least partial engagement between the one or more threads of the translation member and threaded portion of the cutter overmold during reversal of a translation direction of the cutter.

11. The biopsy device of claim 9, the resilient member being configured to maintain at least partial engagement between the one or more threads of the translation member and threaded portion of the cutter overmold during reversal of translation of the cutter from a distal direction to a proximal direction and during reversion of translation of the cutter from the proximal direction to the distal direction.

12. The biopsy device of claim 1, the resilient member being configured to disengage from the cutter overmold for a predetermined range of cutter translation.

13. The biopsy device of claim 1, the cutter overmold being configured to freely rotate relative to the resilient member.

14. The biopsy device of claim 1, the cutter overmold further including a flange, the resilient member being configured to engage the flange.

15. The biopsy device of claim 14, the flange being disposed distally of the threaded portion.

16. A probe for use with a biopsy device, the probe comprising:
(a) a housing;
(b) a needle extending distally from the housing;
(c) a cutter configured to rotate and translate relative to the needle; and
(d) a cutter drive assembly, the cutter drive assembly including a sleeve coaxial with the cutter and configured to drive rotation and translation of the cutter, the sleeve including a threaded portion and a keyed portion, the threaded portion being configured to drive translation of the cutter, the keyed portion being configured to drive rotation of the cutter,
a portion of the housing extending into a hollow interior of the outer housing to engage at least a portion of the sleeve when the sleeve is in a distal position and a proximal position.

17. The probe of claim 16, the portion of the housing extending into the hollow interior of the outer housing including an elongate strip, the elongate strip being configured to bend in response to movement of the sleeve within the housing.

18. The probe of claim 17, the sleeve including a flange and a distal zero pitch portion, the elongate strip being configured to alternatingly engage the flange and the distal zero pitch portion to maintain engagement between the threaded portion and a portion of the cutter drive assembly.

19. The probe of claim 18, the flange and the distal zero pitch portion being separated by a first length, the threaded portion defining a second length, the first length corresponding to the second length.

20. A probe for use with a biopsy device, the probe comprising:
(a) a housing;
(b) a needle extending distally from the housing;
(c) a cutter configured to rotate and translate relative to the needle;
(d) a cutter drive assembly, the cutter drive assembly including a sleeve coaxial with a longitudinal axis defined by the cutter and configured to drive rotation and translation of the cutter, the sleeve including a threaded portion and a keyed portion, the threaded portion being configured to drive translation of the cutter, the keyed portion being configured to drive rotation of the cutter; and
(e) a reversing mechanism mechanically grounded to a portion of the housing, the reversing mechanism being configured to bias the sleeve in a predetermined direction parallel to the longitudinal axis of the cutter.
